(19) United States
(12) Patent Application Publication
(10) Pub. No.: US 2026/0007449 A1
(43) Pub. Date: Jan. 8, 2026

(54) ENERGY SUPPLY DEVICE

(71) Applicant: Karl Storz SE & Co. KG, Tuttlingen (DE)

(72) Inventors: Thomas HINDING, Tuttlingen (DE); Jérôme PIERRE, Tuttlingen (DE)

(21) Appl. No.: 19/181,492

(22) Filed: Apr. 17, 2025

(30) Foreign Application Priority Data
Apr. 18, 2024 (DE) 10 2024 110 877.0

(51) Int. Cl. A61B 18/12; A61B 17/00; A61B 18/00

(52) U.S. Cl. CPC A61B 18/1206; A61B 2017/0019; A61B 2018/00732; A61B 2018/1286

(57) ABSTRACT

An energy supply device for an electrosurgical instrument provides several operational phases. In a first operating phase for igniting a plasma at the electrosurgical instrument, several pulse width modulated (PWM) stages are controlled synchronously to generate a rectangular voltage waveform. In a second operating phase to maintain the plasma, the several PWM stages are controlled individually and, if necessary, with multiple switching operations per period of the output signal to be generated.

### CROSS REFERENCE TO RELATED APPLICATIONS

This application claims the benefit of priority under 35 U.S.C. § 119 of German Application 10 2024 110 877.0, filed Apr. 18, 2024, the entire contents of which are incorporated herein by reference.

### TECHNICAL FIELD OF THE INVENTION

The present invention relates to an energy supply device (also known as a power supply device) for an electrosurgical instrument. The present invention further relates to an electrosurgical system having such an energy supply device and to a method for providing a high-frequency supply voltage for an electrosurgical instrument.

### BACKGROUND OF THE INVENTION

In medical procedures using minimally invasive endoscopy, fast and reliable tissue sealing is of great importance, since even a very small amount of blood can obstruct a surgeon's view. This could make the procedure more difficult or even impossible. To seal the tissue, the relevant tissue can be heated and sealed using a high-frequency current, for example, using an appropriate electrosurgical instrument. In this way, any bleeding can be stopped very quickly.

Electrosurgical systems, for example, can be used for such applications. For example, an electrosurgical system may include a high-frequency generator that generates a high-frequency signal that can be applied to a high-frequency electrode. During a high-voltage discharge at this electrode, a plasma is formed, which can be used for medical treatment such as stopping bleeding (coagulation).

The operation of such an electrosurgical system can be divided into two phases. In a first operating phase (ignition phase) it is first necessary to ignite the plasma at the high-frequency electrode. This requires a relatively high electrical power. In the subsequent further phase, the ignited plasma must be maintained. In this further phase, a significantly lower power than in the ignition phase is sufficient and desired, for example in order to keep the energy input at the treatment site as low as possible. However, it is necessary to control the electrical power as precisely as possible during treatment.

Against this background, an efficient, safe and cost-effective energy supply for such an electrosurgical system is desirable. In particular, a device is desirable which can provide the required high-frequency voltage for igniting and operating an electrosurgical instrument in the simplest possible manner and with the required precision.

### SUMMARY OF THE INVENTION

The present invention provides an energy supply device for an electrosurgical instrument, an electrosurgical system and a method for providing a high-frequency supply voltage for an electrosurgical instrument having features according to the invention. Further advantageous embodiments are disclosed in the description, claims and figures.

According to a first aspect, the present invention provides an energy supply device for an electrosurgical instrument. The energy supply device comprises several PWM (pulse width modulated) stages, a transformer and a control device.

Each PWM stage comprises an input terminal and an output terminal. The PWM stages are configured to be electrically coupled to a DC voltage source at their input terminal. The output terminals of the several PWM stages are electrically coupled to each other at a common node. In other words, the several PWM stages feed their output signals to this common node. The transformer comprises a primary side and a secondary side. A first connection point of the primary side of the transformer is electrically coupled to the common node of the several PWM stages. The second connection point of the primary side of the transformer can be connected to a reference potential.

The transformer is further configured to provide a supply voltage for the electrosurgical instrument between a first connection point and a second connection point of the secondary side. In this way, an electrical output voltage that is galvanically isolated from the DC voltage source can be provided to supply the electrosurgical instrument.

The control device is configured to provide control signals for the several PWM stages. In particular, the control device can distinguish between at least two operating modes. In a first operating mode, the control device can control the several PWM stages synchronously. In other words, the control device provides the same control signal to all PWM stages. In a second operating mode, the control device can control the several PWM stages at different times. In other words, the control device provides individual control signals to the various PWM stages, wherein the edges for switching on and off in the control signals for the individual PWM stages can be provided at different times.

According to a further aspect, an electrosurgical system is provided. The electrosurgical system comprises an electrosurgical instrument and an energy supply device according to the invention. The electrosurgical instrument comprises at least one electrode at which a plasma can be generated.

According to a further aspect, a method for providing a high-frequency supply voltage for an electrosurgical instrument is provided. The high-frequency supply voltage can be provided in particular by means of an energy supply device according to the invention, in particular an energy supply device according to the first aspect. The method comprises a step for synchronously controlling the several PWM stages in a first operating phase. This first operating phase may be intended to ignite a plasma at the electrosurgical instrument. Furthermore, the method comprises a step for controlling the several PWM stages in a staggered manner in a second operating phase. This second phase of operation may be intended to maintain the plasma at the electrosurgical instrument. In particular, the second operating phase may follow immediately after the first operating phase.

The present invention is based on the finding that the operation of an electrosurgical instrument is generally divided into at least two phases. In a first operating phase, during which a plasma is first to be ignited at an electrode of the electrosurgical instrument, a relatively high electrical power must be provided. In a subsequent second operating phase, in which this plasma is to be maintained, a lower electrical power is required. However, it is desirable to control the electrical power as precisely as possible in the second operating phase during maintenance. During the first operating phase (ignition phase), however, the requirements for electrical power control are lower than in the second operating phase.

Based on this finding, it is therefore an idea of the present invention to create an energy supply for such an electrosurgical instrument which, on the one hand, can provide the electrical power required for igniting the plasma and, on the other hand, can also ensure the control accuracy desired while maintaining the plasma. In particular, these requirements can be realized according to the inventive approach by a particularly compact, efficient and thus also cost-effective design.

For this purpose, it is intended to provide the electrical power for the operation of the electrosurgical instrument both during the first operating phase for igniting the plasma and during the second operating phase for maintaining the plasma by means of the same electrical circuit. In particular, a circuit concept with several parallel PWM stages is provided for this purpose. Each of these PWM stages can be controlled by means of a control signal, in particular a pulse width modulated (PWM) control signal.

During a first operating phase for igniting the plasma, all PWM stages can be controlled based on a common PWM signal. This means that all PWM stages are switched jointly. This results in a relatively small number of switching operations compared to the second operating phase described below. A rectangular voltage signal is generated as the output signal. Such a control can generate an output signal with a relatively high efficiency. This good efficiency makes it possible to provide a relatively high output power with comparatively low circuit complexity or smaller-sized circuit components.

In the subsequent second operating phase to maintain the plasma, the individual PWM stages are each controlled with individual PWM signals, wherein these individual PWM signals can fall below each other in the switching times for the respective PWM stages. The individual control signals for the PWM stages can also provide for several switching on and off operations per period of the high-frequency output signal. In this way, it is possible to generate an output signal of which the signal shape can very well approximate a sinusoidal shape. Such voltage waveforms are easier to measure and control during the ignition phase than rectangular voltage waveforms. In this way, the electrical power can be very well monitored and controlled during this operating phase.

This approach, in which the same circuit components, in particular the same PWM stages, are controlled in different ways in the two operating phases, makes it possible, on the one hand, to operate the circuit in a mode with high efficiency during the ignition of the plasma and thus to be able to provide the high electrical power required for the ignition of the plasma.

In addition, by changing the control in the second operating phase to maintain the plasma, very good control of the electrical power can be achieved. Since during this second operating phase the power requirement for maintaining the plasma is lower than the power requirement for igniting the plasma, the lower efficiency of the control in the second operating phase can be accepted. Consequently, the energy supply for the operation of an electrosurgical instrument can be realized with a single circuit arrangement, wherein this circuit arrangement can be made significantly lower and thus also smaller and more cost-effectively due to the very efficient operation during the ignition of the plasma than would be the case if only a single control were provided according to the second operating mode.

According to one embodiment, the control device is configured to control the several PWM stages in the second operating mode with a clock rate which is greater than the clock rate in the first operating mode. In particular, in the second operating mode, several switching on and off operations can be provided in the control signals per period of the high-frequency output signal. This allows the signal shape of the output voltage to be very close to a desired sinusoidal shape

According to one embodiment, the control in the second operating mode comprises a multi-phase PWM control. In such a multi-phase PWM control, the individual PWM stages are each controlled with a common clock frequency. However, the switch-on and switch-off times of the individual PWM stages can differ from one another. The duty cycles for the control signals of the individual PWM stages may also be different. Such a multi-phase PWM control allows a sinusoidal voltage curve to be very well approximated as the common output signal of the PWM stages.

According to one embodiment, an amplitude of the supply voltage for the electrosurgical instrument, which is provided between the first connection point and a second connection point of the secondary side of the transformer, is greater than an electrical input voltage of the DC voltage source, which is provided at the input terminals of the PWM stages and supplies the PWM stages with electrical energy. In other words, the circuit arrangement according to the invention for supplying energy to the electrosurgical instrument outputs an electrical voltage which is (significantly) greater than the input voltage of this circuit arrangement. This makes it possible to dimension the input-side components, especially the PWM stages, to a relatively low electrical voltage. This allows components to be selected which have a small installation volume and which are also available at low cost. The downstream transformer can then be provided, among other things, to increase the electrical voltage to the level required for the operation of the electrosurgical instrument.

According to one embodiment, the input voltage of the DC voltage source is less than or equal to 60 volts. In particular, the input voltage of the DC voltage source, which is provided at the inputs of the PWM stages, can be in the protective extra-low voltage range. In this way, the energy supply device for the electrosurgical instrument can be operated with a safe input voltage. This can prevent a potential danger, for example for a user, from an electric shock or similar. The provision of such an electrical DC voltage can be provided, for example, by means of a separate, external component, such as a corresponding power supply unit or the like. In particular, a galvanically isolating power supply unit can be provided, which can further increase safety.

According to one embodiment, the energy supply device comprises a coupling capacitor. The coupling capacitor can be arranged between the common node of the several PWM stages and the first connection point of the primary side of the transformer. In particular, the coupling capacitor can be adapted for (or: to) the electrical power to be transmitted and/or the frequency of the transmitted electrical signal. Such a coupling capacitor can, for example, be used to decouple DC voltage components or a DC voltage offset for the electrical AC voltage applied to the transformer.

According to one embodiment, the control device is configured to determine an electrical input current from the DC voltage source to the input terminals of the PWM stages. Any suitable methods and components can be provided for this purpose. For example, the electrical input current can be determined using one or more current sensors. In such a configuration, the control device can control the several PWM stages using the determined input current. For example, if the input voltage is known or also determined, the electrical power can be determined from the input current. Thus, based on the determined input current, the electrical power for the electrosurgical instrument can be subjected to open-loop or closed-loop control.

Furthermore, it is also possible, for example, to detect the ignition of the plasma using the determined input current. Thus, after the plasma has been ignited, it is possible to switch from the first operating mode to the second operating mode. Furthermore, it is possible, for example, to detect an (unintentional) extinction of the plasma from the electrical input current. In such a case, for example, it is possible to (automatically) switch back to the first operating phase in order to ignite the plasma again. Of course, the determined electrical input current can also be used for any other applications.

According to one embodiment, the control device is configured to control the several PWM stages in the first operating mode if no plasma is detected at an electrosurgical instrument connected to the output terminal. For example, in a start-up phase it can initially be assumed that no plasma exists yet and therefore the plasma should be ignited. If necessary, the ignition of the plasma can also be triggered by a manual input, for example by a user. However, any suitable detection methods, such as monitoring an electrical current or electrical power, are also possible to determine whether a plasma is present or not. If there is no plasma, the system can automatically switch to the first operating mode to ignite the plasma.

According to one embodiment, the control device is configured to control the several PWM stages in the second operating mode if plasma is detected at an electrosurgical instrument connected to the output terminal. For this purpose, any suitable method, such as monitoring an electrical current or electrical power, can be used to detect a stable plasma at the electrosurgical instrument.

According to one embodiment, the control device is configured to switch from the first operating mode to the second operating mode after a predetermined period of time. For example, the energy supply can initially be carried out in the first operating mode for a fixed predetermined period of time in order to enable the plasma to be ignited. After this period has elapsed, the system can then automatically switch to the second operating mode to maintain an ignited plasma. The predetermined time period after which the switch from the first operating mode to the second operating mode takes place can, for example, be a maximum of 500 ms, for example 200 ms, or a time period deviating therefrom, for example 100 ms, 300 ms, 400 ms or any other suitable time period.

According to one embodiment, the control device comprises an FPGA (Field Programmable Gate Array). Such an FPGA can be configured to generate and provide the control signals for the several PWM stages. For example, each output terminal of the FPGA can provide a control signal for a PWM stage. In principle, however, depending on the application, any other suitable circuit concepts as well as other suitable components or circuits for generating the control signals for the PWM stages are possible.

According to one embodiment, the several PWM stages each comprise a half-bridge. Each half-bridge can comprise two semiconductor switching elements. For example, a first semiconductor switching element may be provided between an input terminal of a PWM stage and an output terminal of the PWM stage, and a second semiconductor switching element may be provided between the output terminal of the PWM stage and a reference potential. Alternatively, a first semiconductor switching element may be provided, for example, between a positive terminal of the DC voltage source and the output terminal, and a second semiconductor switching element may be provided between the output terminal and a negative terminal of the DC voltage source. Depending on the application, however, different circuit concepts for the individual PWM stages are also possible.

According to one embodiment, the control device is configured to control the several PWM stages each with a clock frequency of at least 300 kHz. Such high frequencies generally do not cause any nerve irritation in the patient and are therefore particularly suitable. In principle, however, other frequencies are also possible, especially frequencies of more than 300 kHz, for example 500 kHz, 800 kHz, 1 MHz, 2 MHz or similar.

The above embodiments and developments can be combined with each other as desired, insofar as appropriate. Further embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic forms of the invention.

Further features and advantages of the invention are explained below with reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of an electrosurgical system according to an embodiment;

FIG. 2 is a schematic representation of a basic circuit diagram for an energy supply device of an electrosurgical system according to an embodiment;

FIG. 3 is a timing diagram illustrating the control signals in a first operating mode of the energy supply device according to an embodiment;

FIG. 4 is a timing diagram illustrating the control signal in a second operating mode of the energy supply device according to an embodiment; and

FIG. 5 is a flowchart of a potential basis for a method for providing a high-frequency supply voltage for an electrosurgical instrument according to an embodiment.

### DESCRIPTION OF EMBODIMENTS

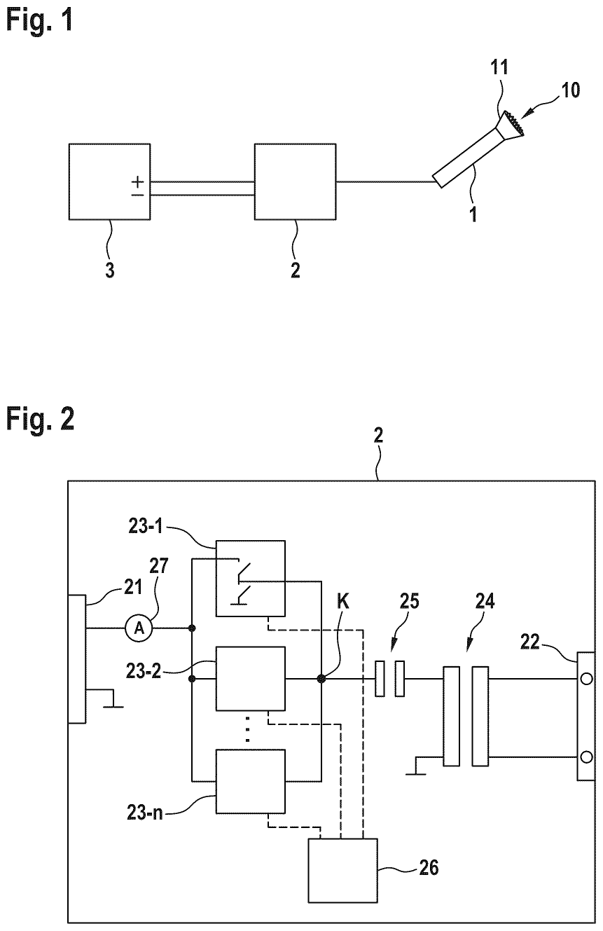

Referring to the drawings, FIG. 1 shows a schematic representation of a block diagram of an electrosurgical system according to one embodiment. The electrosurgical system comprises an electrosurgical instrument 1, in particular an electrosurgical instrument 1 for generating a plasma 10. The plasma 10 can be generated here at one electrode 11 or between two electrodes 11. Such a plasma 10 can, for example, be used to seal tissue in a patient in order to stop bleeding, for example. In principle, however, any other suitable applications are also possible. In particular, the electrosurgical instrument 1 may be a medical instrument for minimally invasive endoscopy.

Electrical energy is required for the operation of the electrosurgical instrument 1 and in particular for the generation of the plasma 10. This electrical energy can be provided by an energy supply device 2. The electrical energy provided by the energy supply device 2 may in particular be a high-frequency electrical voltage. In particular, the high-frequency electrical voltage may have a frequency of at least 300 kHz. For example, the high-frequency electrical voltage can have a frequency of 300 kHz, 400 kHz, 500 kHz, 700 kHz, 1 MHz or 2 MHz. If necessary, the frequency can also be varied or adjusted within a predefinable frequency range. For example, the frequency can be set in a range between 300 kHz and 600 kHz. However, depending on the application, other frequencies or frequency ranges are also possible.

The voltage level of the high-frequency electrical voltage can be set to any suitable value that is suitable for igniting or maintaining the plasma 10. For example, the electrical voltage can have an effective value of 100 V or more. However, depending on the application and configuration of the electrosurgical instrument 1, higher electrical voltages of 200 V, 500 V, 1 kV or more may also be provided. In particular, during operation of the electrosurgical instrument 1, the electrical power converted in the plasma 10 can be adjusted or limited by a suitable control. For this purpose, for example, a closed-loop or open-loop control based on an electrical current can also be provided. In this case, the electrical voltage can also be adjusted in such a way that the electrical current or the electrical power converted in the plasma is set or limited to a predetermined value. Depending on the application, other suitable parameters for open-loop or closed-loop control are also possible.

To supply energy to the electrosurgical instrument 1, the energy supply device 2 can provide the high-frequency electrical voltage for operating the electrosurgical instrument 1. For example, the energy supply device 2 can generate the required high-frequency electrical voltage from a DC voltage provided on the input side of the energy supply device 2. This input DC voltage can be provided, for example, by a DC voltage source 3. This DC voltage source 3 can, for example, be a power supply unit which generates and provides a suitable DC voltage from an electrical AC voltage of a low-voltage energy supply network with a voltage level of, for example, 230 V AC. In particular, galvanic isolation may be provided between the input and the output of the power supply unit. This ensures additional safety for the user against electric shock even in the event of a possible fault, such as an insulation fault.

The electrical DC voltage provided by the DC voltage source 3 can be provided as input voltage of the energy supply device 2. In particular, this input voltage for the energy supply device 2 can have a protective extra-low voltage, such as an electrical direct voltage of maximum 60 V. For example, the input voltage can be 48 V, 24 V, 12 V or 5 V. Of course, any other suitable voltage values are also possible. The input DC voltage for the energy supply device 2 may preferably have a voltage level which is smaller than an amplitude of the high-frequency output voltage for supplying energy to the electrosurgical instrument 1. In other words, the energy supply device 2 not only converts the input DC voltage into a high-frequency AC voltage, but also increases the voltage level. Due to the fact that the input DC voltage is lower than the desired output voltage, the input-side components, in particular the components for generating the high-frequency voltage and for current and/or voltage control, can be configured for the low input-side voltage level.

FIG. 2 shows a schematic representation of a basic circuit diagram for an energy supply device 2 according to an embodiment. The energy supply device 2 can, for example, have an input terminal 21. At this input terminal 21, for example, an input-side DC voltage, such as the DC voltage from the DC voltage source 3, can be provided. Furthermore, the energy supply device 2 can have an output terminal 22. For example, the electrosurgical instrument 1 can be connected to this output connection 22.

The energy supply device 2 may comprise several PWM stages 23-i. The PWM stages 23-i are each connected on the input side to the input terminal 21 of the energy supply device 2. The outputs of the PWM stages 23-i are each connected to a common node K. The PWM stages 23-i can be controlled by means of suitable control signals from a control device 26. For this purpose, the control device 26 can generate a pulse width modulated (PWM) clock signal for each PWM stage 23-i and provide it to the respective PWM stage 23-i. For the calculation and generation of these PWM signals, an FPGA can be provided in the control device 26, for example. In principle, however, the generation of the PWM signals can also be realized in any other suitable way. The generation and properties of the PWM signals are explained in greater detail below.

Any suitable circuit structures can be provided in the individual PWM stages 23-i, whereby switching elements are opened or closed depending on the state of the respective control signal from the control device 26. For example, in each PWM stage 23-i, a half-bridge consisting of a series connection of two semiconductor switching elements can be provided. For example, a first semiconductor switching element M1 may be provided between a positive connection point of the input terminal 21 and an output terminal of the respective PWM stage 23-i, and a second semiconductor switching element M2 may be provided between the output terminal of the PWM stage 23-i and a reference potential or a negative connection point of the input terminal 22. In such a configuration, for example, when the clock signal is logical 1, the first semiconductor switching element M1 can be closed and the second semiconductor switching element M2 can be opened, so that the output terminal of the respective PWM stage 23-i is connected to the positive connection point of the input terminal 21.

Conversely, if the clock signal is logical 0, the first semiconductor switching element M1 can be opened and the second semiconductor switching element M2 can be closed, so that the output terminal of the respective PWM stage 23-i is connected to the negative connection point of the input terminal 21 or the reference potential. When the switching states change, a dead time can be provided between the opening of a semiconductor switching element M1, M2 and the closing of the complementary semiconductor switching element M2, M1 in order to avoid a possible short circuit. However, it is understood that in addition to the circuit configuration of a PWM stage 23-i described here, any other suitable circuit structures may also be possible.

The node K, which is connected to the output terminal of the PWM stages 23-i, is connected, if necessary via a coupling capacitor 25, to a connection point of the primary side of a transformer 24. The second connection point of the primary side of the transformer 24 can be connected to a reference potential or the negative connection point of the input terminal 21. The secondary side connection points of the transformer 24 can be connected to the output terminal 22 of the energy supply device 2.

It is understood that the circuit of the energy supply device 2 described here is basically only to be understood schematically to illustrate the basic principle for generating a high-frequency output voltage from an input DC voltage. Depending on the application, any additional components, such as frequency filters or similar, can of course also be provided. In particular, voltage sensors and/or current sensors, such as the current sensor 27 shown in FIG. 2, can also be provided to detect the input current.

Several operating phases are possible for the operation of the electrosurgical instrument 1. For example, during a first operating phase (ignition phase), a plasma 10 can first be ignited at the electrode(s) 11 of the electrosurgical instrument 1. This usually requires a relatively high amount of electrical energy. Furthermore, this plasma 10 can be maintained during a second operating phase. To maintain the plasma 10, less electrical energy is required than is needed to ignite the plasma 10. On the other hand, in order to control this plasma 10 and the electrical energy converted in this plasma 10, the electrical power or the electrical current for the electrosurgical instrument 1 must be controlled as precisely as possible.

In order to take these complementary requirements for the first operating phase for igniting the plasma 10 and for the second operating phase for maintaining the plasma 10 into account, the control device 26 can provide different control of the PWM stages 23-i for the first operating phase and the second operating phase.

For example, in the first operating phase for igniting the plasma 10, all PWM stages 23-i can be controlled simultaneously, i.e., with the same PWM signal. With such a control with an identical PWM signal for all PWM stages 23-i, a rectangular voltage curve corresponding to the PWM signal results at the node K. For a period of a predetermined frequency of the high-frequency alternating voltage at the output 22 of the energy supply device 2, one switch-on process and one switch-off process take place in each PWM stage 23-i.

In the second operating phase for maintaining the plasma 10, however, an individual, different PWM signal can be generated for each PWM stage 23-i. In particular, several switch-on and switch-off processes are also possible for a period corresponding to the frequency of the high-frequency output signal of the energy supply device 2. Accordingly, the number of switching operations in the PWM stages 23-i increases. This increased number of switching operations compared to the first operating phase increases the switching losses, thereby reducing the efficiency of the energy supply device 2.

On the other hand, this type of control allows the signal shape of the high-frequency output voltage to be better controlled and, in particular, better adapted to a sinusoidal voltage curve. Furthermore, this type of control also allows the electrical current or the output power of the energy supply device 2 to be controlled more precisely. In this way, the electrical energy converted in the plasma 10 and thus the energy input into a patient's tissue during treatment by the electrosurgical instrument 1 can be precisely controlled. Since the electrical power required during this second operating phase to maintain the plasma 10 is lower than during the first operating phase to ignite the plasma 10, the associated lower efficiency due to the increased number of switching operations is of minor importance.

To control the individual PWM stages 23-i during the second operating phase to maintain the plasma 10, the control device 26 can execute the PWM signals for the individual PWM stages 23-i, for example, based on the concept of a so-called multi-phase PWM control. However, since the basic concept of such a multi-phase PWM control can correspond to a conventional multi-phase PWM control, a more detailed explanation is unnecessary here.

The first operating phase for igniting the plasma 10 may, for example, be carried out for a predetermined period of time, for example 200 ms, 500 ms or any other suitable period of time. Subsequently, if necessary, the system can automatically transition to the second operating phase to maintain the plasma 10. Furthermore, it is also possible, for example during the first operating phase, to determine the electrical power, for example by monitoring an electrical current, for example by monitoring the input current by means of a current sensor 27, and from this to conclude that the plasma 10 has been successfully ignited. Since the electric current will increase significantly after the ignition of the plasma 10, the time at which the plasma 10 is ignited can be determined from monitoring the current curve. Thus, after the detection of the successful ignition of the plasma 10, the second operating phase can be entered.

While maintaining the plasma 10 in the second operating phase, any suitable operating parameters, such as an electrical current, such as an input current, may be detected and monitored based on the (output) sensor values of the current sensor 27. In this way, the electrical energy converted in the plasma 10 can be controlled and, in particular, limited.

Furthermore, it is also possible, for example, by monitoring operating parameters such as the input current, to detect an extinction of the plasma 10. If such an extinction of the plasma 10 is detected, it is possible to return to the first operating phase for igniting the plasma 10. Additionally or alternatively, however, manual triggering to ignite the plasma 10 through the first operating phase is also possible.

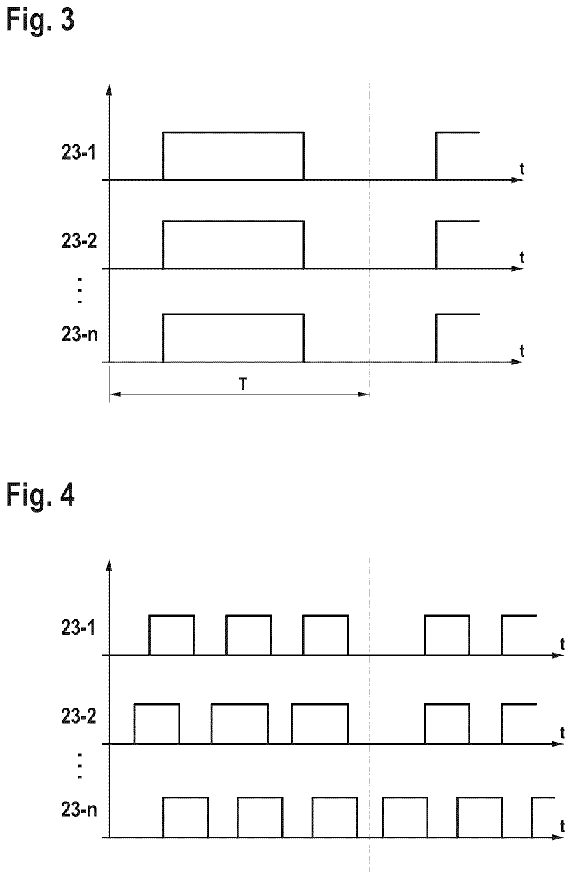

FIG. 3 shows a schematic representation of timing diagrams for control signals of the PWM stages 23-i in a first operating phase. As can be seen here, the individual PWM stages 23-i are each supplied with the same control signal, i.e., with control signals which switch at identical times.

FIG. 4 shows a schematic representation of timing diagrams for the control of PWM stages 23-i in a second operating phase. As can be seen here, the individual PWM stages 23-i are each supplied with individual control signals. The switching operations in the individual PWM stages 23-i can take place at different times. In addition, several switch-on and switch-off operations per PWM stage 23-i can be provided for a period T of the high-frequency output signals.

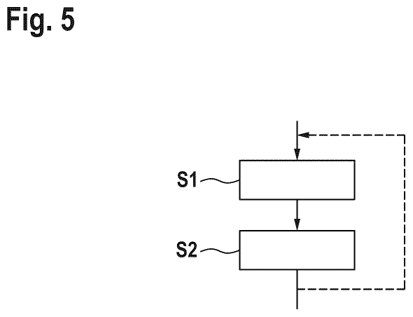

FIG. 5 shows a flowchart of a potential basis for a method for providing a high-frequency supply voltage for an electrosurgical instrument according to an embodiment. The method can be implemented, for example, in a previously described energy supply device 2. Thus, all statements made previously in conjunction with the energy supply device 2 also apply to the method described below. Conversely, the energy supply device 2 can also be configured in any suitable manner in order to implement the method described below.

In a step S1, several PWM stages 23-i are first controlled synchronously. This first operating phase serves, as already explained, to ignite a plasma 10 on the electrosurgical instrument 1.

After this first operating phase, in a step S2 the several PWM stages can be operated in a second operating phase in which the several PWM stages are controlled in a staggered manner. This second operating phase serves, as also previously explained, to maintain the plasma 10. In particular, in this second operating phase, several switch-on and switch-off operations can be provided for a period T of a high-frequency output signal to be output.

If the plasma 10 goes out during operation, it is possible to switch back to the first operating phase, in particular automatically.

In summary, the present invention relates to the energy supply of an electrosurgical instrument. Several operational phases are provided here. In a first operating phase for igniting a plasma at the electrosurgical instrument, several PWM stages are controlled synchronously to generate a rectangular voltage waveform. In a second operating phase to maintain the plasma, the several PWM stages are controlled individually and, if necessary, with multiple switching operations per period of the output signal to be generated.

The drawings, the description, and the claims contain numerous features in combination. It goes without saying that the above-mentioned features can be used not only in the combination specified in each case, but also in other combinations or on their own, without departing from the scope of the present invention. Energy supply device for an electrosurgical instrument. Several operational phases are provided here. In a first operating phase for igniting a plasma at the electrosurgical instrument, several PWM stages are controlled synchronously to generate a rectangular voltage waveform. In a second operating phase to maintain the plasma, the several PWM stages are controlled individually and, if necessary, with multiple switching operations per period of the output signal to be generated.

While specific embodiments of the invention have been shown and described in detail to illustrate the application of the principles of the invention, it will be understood that the invention may be embodied otherwise without departing from such principles.

What is claimed is:

1. An energy supply device for an electrosurgical instrument, the energy storage device comprising:
a plurality of pulse width modulated (PWM) stages, each having an input terminal and an output terminal, wherein each PWM stage is configured to be electrically coupled to a DC voltage source at the input terminal, and wherein the output terminals of the plurality of PWM stages are electrically coupled to one another at a common node;
a transformer comprising a primary side and a secondary side, wherein a first connection point of the primary side of the transformer is electrically coupled to the common node of the plurality of PWM stages, and wherein the transformer is configured to provide a supply voltage for the electrosurgical instrument between a first connection point and a second connection point of the secondary side; and
a control device which is configured to provide control signals for the plurality of PWM stages, wherein the control device is configured to control the plurality of PWM stages synchronously in a first operating mode and to control the plurality of PWM stages at different times in a second operating mode.

2. The energy supply device according to claim 1, wherein the control device is configured to control the plurality of PWM stages in the second operating mode with a clock rate which is greater than the clock rate in the first operating mode.

3. The energy supply device according to claim 1, wherein the control in the second operating mode comprises a multi-phase PWM control.

4. The energy supply device according to claim 1, wherein an amplitude of the supply voltage for the electrosurgical instrument, between the first connection point and a second connection point of the secondary side of the transformer, is greater than an electrical input voltage of the DC voltage source, which is electrically coupled to the input terminals of the PWM stages.

5. The energy supply device according to claim 1, wherein the input voltage of the DC voltage source is less than or equal to 60 volts.

6. The energy supply device according to claim 1, further comprising a coupling capacitor, which is arranged between the common node point of the plurality of PWM stages and the first connection point of the primary side of the transformer.

7. The energy supply device according to claim 1, wherein the control device is configured to determine an electrical input current from the DC voltage source to the input terminals of the PWM stages and to control the plurality of PWM stages using the determined input current.

8. The energy supply device according to claim 1, wherein the control device is configured to control the plurality of PWM stages in the first operating mode if no plasma is detected at an electrosurgical instrument connected to the output terminal.

9. The energy supply device according to claim 1, wherein the control device comprises a field programmable gate array (FPGA) which is configured to generate and provide the control signals for the plurality of PWM stages.

10. The energy supply device according to claim 1, wherein the control device is configured to change from the first operating mode to the second operating mode after a predetermined period of time.

11. The energy supply device according to claim 1, wherein the plurality of PWM stages each comprise a half-bridge with two semiconductor switching elements.

12. The energy supply device according to claim 1, wherein the control device is configured to control the plurality of PWM stages each with a clock frequency of at least 300 kHz.

13. An electrosurgical system comprising:
an electrosurgical instrument, which comprises at least one electrode configured to generate a plasma; and
an energy power supply device, the energy storage device comprising: a plurality of pulse width modulated (PWM) stages, each having an input terminal and an output terminal, wherein each PWM stage is configured to be electrically coupled to a DC voltage source at the input terminal, and wherein the output terminals of the plurality of PWM stages are electrically coupled to one another at a common node; a transformer comprising a primary side and a secondary side, wherein a first connection point of the primary side of the transformer is electrically coupled to the common node of the plurality of PWM stages, and wherein the transformer is configured to provide a supply voltage for the electrosurgical instrument between a first connection point and a second connection point of the secondary side; and a control device which is configured to provide control signals for the plurality of PWM stages, wherein the control device is configured to control the plurality of PWM stages synchronously in a first operating mode and to control the plurality of PWM stages at different times in a second operating mode.

14. The electrosurgical system according to claim 13, further comprising the DC voltage source, which is configured to provide a DC voltage with a predetermined voltage level at the input terminals of the plurality of PWM stages.

15. The electrosurgical system according to claim 14, wherein the control device is configured to control the plurality of PWM stages in the second operating mode with a clock rate which is greater than the clock rate in the first operating mode.

16. The electrosurgical system according to claim 14, wherein the control device is configured to control the plurality of PWM stages in the first operating mode if no plasma is detected at an electrosurgical instrument connected to the output terminal, and wherein the control in the second operating mode comprises a multi-phase PWM control.

17. The electrosurgical system according to claim 14, wherein an amplitude of the supply voltage for the electrosurgical instrument, between the first connection point and a second connection point of the secondary side of the transformer, is greater than an electrical input voltage of the DC voltage source, which is electrically coupled to the input terminals of the PWM stages.

18. The electrosurgical system according to claim 14, further comprising a coupling capacitor, which is arranged between the common node point of the plurality of PWM stages and the first connection point of the primary side of the transformer.

19. The electrosurgical system according to claim 14, wherein the control device is configured to determine an electrical input current from the DC voltage source to the input terminals of the PWM stages and to control the plurality of PWM stages using the determined input current.

20. A method for providing a high-frequency supply voltage for an electrosurgical instrument comprising an energy power supply device, the energy storage device comprising: a plurality of pulse width modulated (PWM) stages, each having an input terminal and an output terminal, wherein each PWM stage is configured to be electrically coupled to a DC voltage source at the input terminal, and wherein the output terminals of the plurality of PWM stages are electrically coupled to one another at a common node; a transformer comprising a primary side and a secondary side, wherein a first connection point of the primary side of the transformer is electrically coupled to the common node of the plurality of PWM stages, and wherein the transformer is configured to provide a supply voltage for the electrosurgical instrument between a first connection point and a second connection point of the secondary side; and a control device which is configured to provide control signals for the plurality of PWM stages, wherein the control device is configured to control the plurality of PWM stages synchronously in a first operating mode and to control the plurality of PWM stages at different times in a second operating mode, the method comprising the steps of:
synchronously controlling the plurality of PWM stages in a first operating phase for igniting a plasma on the electrosurgical instrument; and
controlling the plurality of PWM stages in a staggered manner in a second operating phase for maintaining the plasma at the electrosurgical instrument.
